(19) United States
(12) Patent Application Publication
(10) Pub. No.: US 2011/0082161 A1
(43) Pub. Date: Apr. 7, 2011

(54) POWDERS FOR RECONSTITUTION

(76) Inventors: Lieven Elvire Colette Baert, Mechelen (BE); Jody Firmin Marceline Voorspoels, Reningelst (BE); Filip Rene Irene Kiekens, Beerse (BE)

(21) Appl. No.: 12/997,185

(22) PCT Filed: Jun. 30, 2009
(86) PCT No.: PCT/EP2009/004701

§ 371 Date: Dec. 9, 2010

(30) Foreign Application Priority Data
Jun. 30, 2008 (EP) 08159336.0

(51) Int. Cl. A61K 31/505; A61P 31/18

(52) U.S. Cl. 514/272

(57) ABSTRACT

This invention relates to drinkable formulations prepared from powders for reconstitution comprising etravirine (TMC125) dispersed in certain water-soluble polymers, which can be used in the treatment of HIV infection.

### FIELD OF THE INVENTION

This invention relates to drinkable formulations prepared from powders for reconstitution comprising etravirine (TMC125) dispersed in certain water-soluble polymers, which can be used in the treatment of HIV infection.

### BACKGROUND OF THE INVENTION

The treatment of Human Immunodeficiency Virus (HIV) infection, known as cause of the acquired immunodeficiency syndrome (AIDS), remains a major medical challenge. Current treatment of HIV infection involves using drugs of various classes such as nucleoside reverse transcriptase inhibitors (NRTIs), non-nucleoside reverse transcriptase inhibitors (NNRTIs), nucleotide reverse transcriptase inhibitors (NtRTIs), HIV-protease inhibitors (PIs), entry inhibitors, and integrase inhibitors.

Although effective in suppressing HIV, each of these drugs, when used alone, is confronted with the emergence of resistant mutants. This led to the introduction of combination therapy of several anti-HIV agents with a different activity profile. In particular the introduction of “HAART” (Highly Active Anti-Retroviral Therapy) resulted in a remarkable improvement in anti-HIV therapy. However, none of the currently available drug therapies is capable of completely eradicating HIV. Even HAART can face the emergence of resistance, often due to non-adherence to and non-persistence with antiretroviral therapy. In these cases HAART can be made effective again by replacing one of its components by one of another class. If applied correctly, treatment with HAART combinations can suppress the virus for many years, up to decades, to a level where the outbreak or progress of AIDS is blocked.

One class of HIV drugs used in HAART is that of the NNRTIs, a number of which are currently on the market and others are in various stages of development. One such NNRTI is the compound 4-[[6-amino-5-bromo-2-[(4-cyanophenyl)amino]-4-pyrimidinyl]oxy]-3,5-dimethylbenzonitrile, also referred to as etravirine, R165335, or TMC125. In a growing number of countries, TMC125 is on the market under the tradename “Intelence™”. TMC125 not only shows pronounced activity against wild type HIV, but is remarkably active against mutant strains. This compound, its pharmacological activity, as well as a number of procedures for its preparation have been described in WO 00/27825. Various conventional pharmaceutical dosage forms, including tablets, capsules, drops, suppositories, oral solutions and injectable solutions are mentioned therein.

TMC125 is very insoluble in aqueous media and therefore shows low bioavailability. WO 01/23362 and WO 01/22938 disclose solid dispersions of this compound in water-soluble polymers resulting in improved bioavailability, especially when in the form of powders prepared by spray drying. WO 2007/141308 describes solid dispersions of TMC125 and microcrystalline cellulose in a water-soluble polymer, as well as processes for preparing these dispersions. Current tablet formulations of TMC125 are based on a solid dispersion of TMC125 in hydroxypropyl methylcellulose (HPMC) obtained by spray drying. The spray-dried powder is mixed with further ingredients and compressed to a tablet.

The current dosing regimen of TMC125 is 200 mg twice a day (b.i.d.), administered as two tablets each containing 100 mg, to be taken in at once, preferably two in the morning and two at the end of the day. Because these quantity requirements and the fact that TMC125 is dispersed in a relatively large quantity of water-soluble polymer, oral dosage forms of this drug are inevitably large in size.

Infants and children constitute a growing group of HIV infected patients. Paediatric anti-HIV medication poses particular challenges in that the dose regimens vary to a large extent due to variations in age and body weight (babies—children). Especially in the first year after birth, an infant undergoes rapid changes and body weight increases spectacularly. Because of these rapid changes at young age, dosing of a drug needs to be adjusted frequently and dosage forms need to offer flexibility in dosing. Traditional dosage forms such as pills and capsules lack the dosing flexibility required in paediatric applications. Moreover, these dosage forms are not fit for administration to young children and especially to infants in which case drinkable formulations are the preferred route of administration. These comprise liquid formulations such as syrups as well as dry formulations such as powders for reconstitution in which the drug is distributed in dry form and is converted in liquid form by adding water.

Powders for reconstitution are attractive over liquid oral dosage forms because of their compactness making them more convenient for storage and transport. Incorporating TMC125 in a powder for reconstitution poses particular challenges in that it is poorly soluble in water. Upon addition of water, only a very limited amount of the active ingredient is dissolved and when taken in no effective blood plasma levels are Hence there is a need to provide a powder of reconstitution of TMC125 that upon addition of water results in a drinkable dosage form that results in effective therapeutic concentrations of the active. There is a further need to provide a drinkable dosage form prepared from a powder of reconstitution in which the TMC125 remains in amorphous form and does not crystallize. Additionally, it would be advantageous to provide a drinkable dosage form prepared from a powder of reconstitution that remains stable in that the particles in the drinkable dosage form do not precipitate or only precipitate after a long time. The latter property ensures that the active ingredient does not partly sink to the bottom of the container and partially remains there after drinking or sticks to the walls of the container, which container can be a drinking glass or small bottle. This ensures that the whole dose of the active ingredient is taken in, which is important in the case of anti-HIV drugs where correct dosing is crucial.

It now has been found that the NNRTI TMC125 can be converted into a powder for reconstitution using specific water-soluble polymers. These powders allow a flexible application of the active ingredient and moreover are fit for paediatric applications. The powders for reconstitution of this invention may also be applied in adult patient groups that have difficulty or find inconvenience in swallowing, for example the elderly. The powders for reconstitution of this invention may contain several active ingredients thereby allowing the administration of drug cocktails in one administration. This results in a reduced number of administrations thereby being beneficial in terms of pill burden and drug compliance of the patient.

The powders for reconstitution in accordance with the invention, when mixed with an aqueous medium, in particular, when mixed with water, result in a suspension that is stable in that the active ingredient TMC125 stays in amorphous form, and that upon intake, results in effective blood plasma levels. The TMC125 does not crystallize and remains in its amorphous form, also over longer periods of time. The latter comprises periods up to about six hours, or up to about 4 hours, or up to about 2 hours, or up to about 1 hour. The resulting drinkable suspensions moreover are physically stable in that the suspended particles do not sink or stick to the side of the container in which they are prepared or stored, also over longer periods of time. These periods can be as those mentioned above. Upon administration, the resulting drinkable suspensions lead to an efficient uptake of the active ingredient TMC125, which in turn results in adequate bioavailability and effective blood plasma levels.

### DESCRIPTION OF THE INVENTION

In one aspect the present invention relates to the use of a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose, to be mixed with water, for the manufacture of a medicament for the treatment of a subject infected with HIV. In one embodiment said powder is obtained by spray-drying. These powders can be referred to as powders for reconstitution in that they can be mixed with aqueous media, as described hereinafter, to provide liquid formulations, in particular drinkable liquid formulations.

In a further aspect there is provided a method of treating a patient infected with HIV, said method comprising the administration to said patient of a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose, wherein prior to administration the powder is mixed with water. The amount of TMC125 in said powder is preferably an anti-virally effective amount.

In a further aspect there is provided a suspension of amorphous TMC125, or a pharmaceutically acceptable acid-addition salt thereof, and a water-soluble polymer selected from polyvinylpyrrolidone, a copolymer of vinylpyrrolidone and vinyl acetate, a hydroxyalkyl alkylcellulose, in an aqueous medium. Said suspension of amorphous TMC125 can be obtained by adding water to a powder comprising an anti-virally effective amount of TMC125 dispersed in a water-soluble polymer selected from polyvinylpyrrolidone, a copolymer of vinylpyrrolidone and vinyl acetate, a hydroxyalkyl alkylcellulose, In an alternative aspect there is provided a suspension of amorphous TMC125 and a water-soluble polymer selected from polyvinylpyrrolidone, a copolymer of vinylpyrrolidone and vinyl acetate, a hydroxyalkyl alkylcellulose, obtainable or obtained by adding water to a powder comprising an anti-virally effective amount of TMC125 dispersed in a water-soluble polymer selected from polyvinyl-pyrrolidone, a copolymer of vinylpyrrolidone and vinyl acetate, a hydroxyalkyl alkylcellulose,

In another aspect, there is provided a method for the treatment of a subject infected with HIV, said method comprising the administration of a suspension of amorphous TMC125, or a pharmaceutically acceptable acid-addition salt thereof, and a water-soluble polymer selected from polyvinylpyrrolidone, a copolymer of vinylpyrrolidone and vinyl acetate, a hydroxyalkyl alkylcellulose, in an aqueous medium.

The invention furthermore provides a method of, or alternatively, a process for preparing a suspension of amorphous TMC125, or a pharmaceutically acceptable acid-addition salt thereof, and a water-soluble polymer selected from polyvinylpyrrolidone, a copolymer of vinylpyrrolidone and vinyl acetate, a hydroxyalkyl alkylcellulose, in an aqueous medium, said method or process comprising adding water to TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose and a poloxamer. In one embodiment the suspension of amorphous TMC125 is prepared by adding water to a powder of TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose, and a poloxamer.

In a further aspect the present invention concerns a drinkable formulation obtainable by mixing an aqueous phase with a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose.

In a further aspect the present invention concerns a drinkable formulation comprising particles, wherein the particles comprise TMC125, or a pharmaceutically acceptable acid-addition salt thereof, dispersed in a water-soluble polymer selected from poly-vinylpyrrolidone, a copolymer of vinylpyrrolidone and vinyl acetate, a hydroxyalkyl alkylcellulose, suspended in an aqueous phase. The drinkable formulations may take the form of a suspension of said particles comprising TMC125 dispersed in a water-soluble polymer.

The aqueous phase can be water, optionally containing a further ingredients such as a flavors, a colorants, a sweeteners, a taste making agents, and the like. Sweeteners can be sugars, sorbitol and the like agents. Flavors also include acids that provide a pleasant taste such as citric acid. Other agents that can be used are preservatives and thickeners.

When contacting the powders of reconstitution of the present invention with an aqueous medium, in particular with water, part of the water-soluble polymer may go into solution whereby the aqueous phase contains part of the water-soluble polymer in solution. This may additionally help to stabilize the drinkable formulation. Where the powder for reconstitution additionally contains ingredients such as microcrystalline cellulose, these ingredients may become co-suspended and have a stabilizing effect on the suspensions.

It has been found that when contacting the powders for reconstitution, in particular those prepared by spray drying, comprising TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose, with aqueous media, in particular with water, in the resulting particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1, in particular is about 2:1. This can be measured by filtering off the particles and measuring said weight ratios. Said weight ratio is attained independently of the weight ratio of the powder that is mixed with the aqueous phase, as long as said ratio is higher than or equal to 2:1 (TMC125:polymer).

The resulting particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1, or in particular in the range of about 2:1, constitute a further aspect of the present invention. These are stable particles that upon intake result in adequate absorption of the active ingredient and concomitant effective blood plasma levels.

The present invention also concerns a process for preparing a drinkable formulation as specified herein, wherein water is mixed with a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, a hydroxyalkyl alkylcellulose.

The term alkyl has its usual meaning as known to those skilled in the art and may include, for example, C₁₋₆alkyl, or C₁₋₄alkyl, or C₁₋₂alkyl. The term C₁₋₆alkyl defines hydrocarbon radicals, preferably saturated hydrocarbon radicals, having from 1 to 6 carbon atoms, such as methyl, ethyl, 1-propyl 2-propyl, 1-butyl, 2-butyl, 2-methyl-2-propyl, 2-methyl-1-propyl. C₁₋₄alkyl has 1-4 carbon atoms and C₁₋₂alkyl has 1-2 carbon atoms

The powders for reconstitution for use in the present invention comprise the active ingredient TMC125 dispersed in particular water-soluble polymers, which may be referred to as a solid dispersion of the active ingredient TMC125, or a pharmaceutically acceptable acid-addition salt thereof, in the particular water-soluble polymers. The latter include polyvinylpyrrolidone (PVP) and copolymers of vinylpyrrolidone and vinyl acetate (PVPCoVA, sometimes also referred to as PVP-VA, or as copovidone); hydroxyalkyl alkylcelluloses, in particular hydroxyC₁₋₄alkyl C₁₋₄alkylcelluloses such as hydroxypropylmethylcellulose (HPMC).

The amount of water-soluble polymer in the solid dispersion of TMC125, in the particular water-soluble polymers may be in the range from about 50% to about 99%, in particular about 60% to about 90%, or about 70% to about 80% or about 73% to about 77%, e.g. about 75%, by weight relative to the total weight of the solid dispersion. The weight:weight ratio of water-soluble polymer to TMC125 in the solid dispersion of TMC125 in the particular water-soluble polymers may be in the range of about 50:1 to about 1:5, or about 20:1 to about 1:1, or about 10:1 to about 1:1, or about 5:1 to about 1:1, more in particular of about 1:2 to about 1:4, for example said weight:weight ratio is about 3:1, or about 2:1. The amount of TMC125 in the solid dispersion of TMC125 in the particular water-soluble polymers may be in the range from about 1% to about 50%, in particular about 5% to about 45%, or about 10% to about 40% or about 20% to about 30%, e.g. about 25%, by weight relative to the total weight of the solid dispersion. The above percentages and ratios apply equally in the instance where a pharmaceutically acceptable acid-addition salt of TMC125 is used. In that instance the percentages and ratios are recalculated in function of the increased molecular weight of the salt used. The above percentages and ratios should therefore be interpreted as relating to TMC125 base or equivalents of TMC125 base.

In one embodiment, the water-soluble polymer has a molecular weight in the range 500 D to 2 MD. The water-soluble polymer may have an apparent viscosity of 1 to 15,000 mPa·s, or of 1 to 5000 mPa·s, or of 1 to 700 mPa·s, or of 1 to 100 mPa·s when in a 2% (w/v) aqueous solution at 20° C.

Particular hydroxyalkyl alkylcelluloses include hydroxyethyl methylcellulose and hydroxypropyl methylcellulose (or HPMC, e.g. HPMC 2910 15 mPa·s; HPMC 2910 5 mPa·s). Particular vinylpyrrolidones include PVP K12, PVP K17, PVP K25, PVP K29-32, PVP K90.

Said HPMC contains sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule.

Hydroxypropyl molar substitution refers to the average number of moles of propylene oxide which have reacted with each anhydroglucose unit of the cellulose molecule. A preferred HPMC is hypromellose 2910 15 mPa·s or hypromellose 2910 5mPa·s, especially hypromellose 2910 15 mPa·s. Hydroxypropyl methylcellulose is the United States Adopted Name for hypromellose (see Martindale, The Extra Pharmacopoeia, 29th edition, page 1435). In the four digit number “2910”, the first two digits represent the approximate percentage of methoxy groups and the third and fourth digits the approximate percentage composition of hydroxypropoxyl groups; 15 mPa·s or 5 mPa·s is a value indicative of the apparent viscosity of a 2% aqueous solution at 20° C.

Copolymers of vinylpyrrolidone and vinyl acetate that may be used include those copolymers wherein the molecular ratio of the monomers vinylpyrrolidone to vinyl acetate is about 1.2 or wherein the mass ratio of the monomers vinylpyrrolidone to vinyl acetate is about 3:2. Such copolymers are commercially available and are known as copovidone or copolyvidone, sold under trademarks Kolima™ or Kollidon VA 64™. The molecular weight of these polymers may be in the range of about 45 to about 70 kD. The K-value, obtained from viscosity measurements may be in the range of about 25 to about 35, in particular the K value may be about 28.

Polyvinylpyrrolidine polymers that may be used are known as povidone (PVP) and are commercially available. They may have a molecular weight that is in the range of about 30 kD to about 360 kD. Examples are PVP K12 (BASF, MW 2000-3000), PVP K17 (BASF, MW=7000-11000) PVP K25 (BASF, MW=28000-34000), PVP K30 (BASF, MW=44000-54000), and PVP K90 (BASF, MW=1000000-1500000), available under the tradename Kollidon™.

The active ingredient TMC125 may be used as such, i.e. as the base form, but may also be used as a pharmaceutically acceptable acid-addition salt form. The pharmaceutically acceptable addition salts are meant to comprise the therapeutically active non-toxic salt forms. The acid addition salt forms can be obtained by treating the base form with appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Of interest are the hydrohalic acid salts, in particular the hydrobromic acid salt.

The TMC125 in the dosage forms of the invention is present in the form of a solid dispersion in a water-soluble polymer. Different types of solid dispersions exist. One type of solid dispersion is where the pharmaceutical agent is molecularly dispersed, substantially homogeneously, throughout the polymer. This is generally described as a “solid solution”. Another type of solid dispersion is where there are islands or clusters of crystalline or semi-crystalline pharmaceutical agent dispersed throughout the polymer. A further type of solid dispersion is where there are islands or clusters of pharmaceutical agent in amorphous form dispersed throughout the polymer. There may also be solid dispersions comprising mixtures of two or more of the above types, for example a solid solution with areas where the pharmaceutical agent is crystalline or semi-crystalline, or where there are islands or clusters of the agent in amorphous form. All these types will be commonly designated hereinafter as “solid dispersions”.

The active ingredient TMC125 may be dispersed more or less evenly throughout the water-soluble polymer or can be dispersed uniformly or homogenously throughout the polymer. In the former instance the active ingredient is dispersed less homogenous throughout the polymer. There may be domains or small regions wherein amorphous, nanocrystalline, microcrystalline or crystalline TMC125, or amorphous, micro-crystalline or crystalline water-soluble polymer, or both, are dispersed more or less evenly in the water-soluble polymer.

Various techniques exist for preparing solid dispersions including ball milling, melt-extrusion, spray-drying, spray freezing, solution-evaporation, and supercritical fluid technologies.

The solution-evaporation process comprises the following steps:
a) dissolving TMC125 and the water-soluble polymer in an appropriate solvent, optionally at elevated temperature;
b) allowing the solvent in the solution obtained in step a) to evaporate, optionally by heating, or optionally under vacuum, or both, until dry material is obtained.

The solution may also be poured onto a large surface so as to form a thin film, and the solvent evaporated there from.

The melt-extrusion process usually comprises the following steps:
a) optionally mixing TMC125 and the water-soluble polymer,
b) optionally blending additives with the thus obtained mixture,
c) heating and compounding the thus obtained blend until one obtains a homogeneous melt,
d) forcing the thus obtained melt through one or more nozzles; and
e) cooling the melt until it solidifies.

The terms “melt” and “melting” should be interpreted broadly. These terms not only mean the alteration from a solid state to a liquid state, but can also refer to a transition to a glassy state or a rubbery state, and in which it is possible for one component of the mixture to get embedded more or less homogeneously into the other. In particular cases, one component will melt and the other component(s) will dissolve in the melt thus forming a solution, which upon cooling may form a solid solution having advantageous dissolution properties.

After preparing the solid dispersions as described hereinabove, the obtained products are milled and optionally sieved. The solid dispersion product may be milled or ground to particles having a particle size of less than 600 μm, or less than 400 μm, or less than 125 μm, or to the particle sizes mentioned hereinafter.

A preferred process to prepare the powders for reconstitution for use in the present invention is by the spray-drying technique. In the spray-drying technique, TMC125 and the water-soluble polymer are dissolved in an appropriate solvent and the resulting solution is then sprayed through the nozzle of a spray dryer whereby the solvent from the resulting droplets is evaporated, usually at elevated temperatures, e.g. by the introduction of hot air.

The amount of water-soluble polymer in the spray dried product may be in the range from about 50% to about 99%, in particular about 60% to about 90%, or about 70% to about 80% or about 73% to about 77%, e.g. about 75%,by weight relative to the total weight of the spray dried product comprising TMC125, water-soluble polymer, and optional excipients. The weight:weight ratio of water-soluble polymer to TMC125 in the spray dried product of TMC125 in the particular water-soluble polymers may be in the range of about 50:1 to about 1:5, or about 20:1 to about 1:1, or about 10:1 to about 1:1, or about 5:1 to about 1:1, for example said weight:weight ratio is about 3:1, or about 2:1. The amount of TMC125 the spray dried product of TMC125 in the particular water-soluble polymers may be in the range from about 1% to about 50%, in particular about 5% to about 45%, or about 10% to about 40% or about 20% to about 30%, e.g. about 25%, by weight relative to the total weight of the spray dried product. The amount of water-soluble polymer in the feed mixture can be calculated based on these percentages and on the amount of solvent used.

An ingredient that may be added to the spray mixture is microcrystalline cellulose (MCC) resulting in a powder of increased density thereby improving properties such as flowability.

The microcrystalline cellulose (MCC) that can be added to the mixture for spray-drying has an average particle size, which is selected such that when mixed into the solution of pharmaceutical agent and water-soluble polymer, the resulting feed mixture is able to pass through the atomizing means into the spray-drying chamber without clogging or blocking the atomizer. As such, the size of the MCC is limited by the particular size of the atomizing means provided on the spray-drying chamber. For example, where the atomizing means is a nozzle, the size of the nozzle bore will affect the size range of the MCC that may be used. The average particle size of the MCC may be in the range of from 5 μm to 50 μm, in particular from 10 μm to 30 μm, e.g. about 20 μm.

Microcrystalline cellulose that can be used comprises the Avicel™ series of products available from FMC BioPolymer, in particular Avicel PH 105™ (20 μm), Avicel PH 101™ (50 μm), Avicel PH 301™ (50 μm);

the microcrystalline cellulose products available from JRS Pharma, in particular Vivapur™ 105 (20 μm), Vivapur™ 101 (50 μm), Emcocel™ SP 15 (15 μm), Emcocel™ 50M 105 (50 μm), Prosolv™ SMCC 50 (50 μm);
the microcrystalline cellulose products available from DMV, in particular Pharmacel™ 105 (20 μm), Pharmacel™101 (50 μm);
the microcrystalline cellulose products available from Blanver, in particular Tabulose (Microcel)™101 (50 μm), Tabulose (Microcel)™103 (50 μm);
the microcrystalline cellulose products available from Asahi Kasei Corporation, such as Ceolus™ PH-F20JP (20 μm), Ceolus™ PH-101 (50 μm), Ceolus™ PH-301 (50 μm), Ceolus™ KG-802 (50 μm).

A particularly preferred microcrystalline cellulose is Avicel PH 105® (20 μm).

The amount of MCC in the spray dried product may be in the range from about 5% to about 25%, in particular about 7.5% to about 20%, or about 10% to about 15% or about 10% to about 12.5%, by weight relative to the total weight of the spray dried product comprising TMC125, water-soluble polymer, MCC and optional excipients. The weight ratio of the amounts of MCC to TMC125 in the spray dried product can be calculated based on these percentages and in particular may be in the range of from about 2:1 to about 1:5, in particular from about 1:1 to 1:7, preferably about 1:2. The amount of MCC in the feed mixture can be calculated based on these percentages and on the amount of solvent used. In view of the desirability of keeping the concentration of pharmaceutical agent in the resulting solid pharmaceutical composition as high as possible, the concentration of MCC is preferably kept as low as possible.

An advantage of using microcrystalline cellulose is that in addition to increasing the density of the resulting solid pharmaceutical composition, it may also function to increase the properties of flowability, compressibility, disintegration and dissolution of the spray-dried solid dispersion of TMC125 and of pharmaceutical compositions derived therefrom.

Particular powders for reconstitution in accordance with this invention are those comprising TMC125/HPMC/MCC 1/0.5/0.5 w/w or TMC125/HPMC/MCC 1/3/0.5 w/w. In others no MCC is present.

The solvent used in the spray drying procedure may be any solvent that is inert towards TMC125 and that is able to dissolve TMC125 and the water-soluble polymer. Suitable solvents include acetone, tetrahydrofuran (THF), dichloromethane, ethanol (anhydrous or aqueous), methanol, and combinations thereof. Of interest are mixtures of ethanol and methylene chloride, in particular mixtures of the latter two solvents wherein the v/v ratio ethanol/methylene chloride is in the range of about 50:50 to about 90:10; or in the range of about 50:50 to about 80:20, e.g. in a 50:50 ratio, or in a 90:10 ratio.

In one embodiment, the solvent is a mixture of dichloromethane and ethanol, the latter in particular being anhydrous ethanol. In another embodiment, the solvent is dichloromethane.

The amount of solvent present in the feed mixture will be such that TMC125 and the water-soluble polymer are dissolved and that the feed mixture has sufficient low viscosity for it to be sprayed. In one embodiment the amount of solid materials in the feed mixture is less than 20%, in particular less than 10%, more in particular less than 5%, the percentages expressing the weighed amount of solid materials to the total volume of the feed mixture.

The solvent is removed from the droplets of the feed mixture by the spray-drying step. Preferably the solvent is volatile, with a boiling point of 150° C. or less, preferably 100° C. or less.

The drying gas may be any gas. Preferably, the gas is air or an inert gas such as nitrogen, nitrogen-enriched air or argon. The temperature of the drying gas at the gas inlet of the spray-drying chamber can be from about 25° C. to about 300° C., or from about 60° C. to about 300° C., or from about 60° C. to about 150° C.

The spray-drying is conducted in a conventional spray-drying apparatus comprising a spray-drying chamber, atomizing means for introducing the feed mixture into the spray-drying chamber in the form of droplets, a source of heated drying gas that flows into the spray-drying chamber through an inlet, and an outlet for the heated drying gas. The spray-drying apparatus also comprises a means for collecting the solid pharmaceutical powder that is produced. The atomizing means can be a rotary atomizer, a pneumatic nozzle, an ultrasonic nozzle or, preferably, a high-pressure nozzle.

Suitable rotary atomizers include those having an air turbine drive operating from a high pressure compressed air source, for example a 6 bar compressed air source, which supplies power to an atomization wheel for atomizing the feed mixture. The atomization wheel may be vaned. Preferably, the rotary atomizer is located in the upper part of the spray-drying chamber, for example in the chamber roof, so that the droplets produced dry and fall to the lower part of the chamber. Typically, rotary atomizers produce droplets that have a size in the range of from about 20 to about 225 μm, in particular from about 40 to about 120 μm, the droplet size depending upon the wheel peripheral velocity.

Suitable pneumatic nozzles (including two-fluid nozzles) comprise those that are located in the upper part of the spray-drying chamber, for example in the chamber roof, and operate in so-called “co-current mode”. Atomization takes place using compressed air such that the air-liquid ratio is in the range of about 0.5-1.0:1 to about 5:1, in particular from about 1:1 to about 3:1, The feed mixture and the atomizing gas are passed separately to the nozzle head, where the atomization takes place. The size of the droplets produced by pneumatic nozzles depends on the operating parameters and can be in the range from about 5 to 125 μm, in particular from about 20 to 50 μm.

Two-fluid nozzles that operate in so-called “counter-current mode” may also be used. These nozzles operate in a similar way to two-fluid nozzles in co-current modes except that they are located in a lower part of the drying chamber and spray droplets upwards. Typically, counter-current two-fluid nozzles generate droplets, which, when dried, produce particles having a size in the ranging from about 15 to about 80 μm.

Suitable ultrasonic atomizer nozzles convert low viscosity liquids into ultra fine sprays. As liquids are pumped through the center of the probe, the liquids are mechanically pulverized into droplets from the vibrating tip. These droplets are larger with low frequency probes and smaller with higher frequency probes.

A preferred atomizer type for use in the invention is the high-pressure nozzle where liquid feed is pumped to the nozzle under pressure. Pressure energy is converted to kinetic energy, and feed issues from the nozzle orifice as a high-speed film that readily disintegrates into a spray as the film is unstable. The feed is made to rotate within the nozzle using a swirl insert or swirl chamber resulting in cone-shaped spray patterns emerging from the nozzle orifice. Swirl insert, swirl chamber and orifice dimensions together with variation of pressure gives control over feed rate and spray characteristics. The size of the droplets produced by high-pressure nozzles depends on the operating parameters and can be in the range from about 5 to 125 μm, in particular from about 20 to 50 μm.

Suitable atomizing means may be selected depending on the desired droplet size, which depends on a number of factors, such as the viscosity and temperature of the feed mixture, the desired flow rate and the maximum acceptable pressure to pump the feed mixture, have on droplet size. After selecting the atomizing means so that the desired average droplet size is obtained for a feed mixture having a particular viscosity, the mixture is admitted to the spray-drying chamber at a particular flow rate.

The solid dispersion produced by the spray drying process, or produced by the other processes described above (such as solution evaporation and melt extrusion), followed by milling and optional sieving, typically comprises particles having an average effective particle size in the range of from about 10 μm to about 150 μm, or about 15 μm to about 100 μm, particularly about 20 μm to about 80 μm, or 30 μm to about 50 μm, for example about 40 μm or about 50 μm. As used herein, the term average effective particle size has its conventional meaning as known to the person skilled in the art and can be measured by art-known particle size measuring techniques such as, for example, sedimentation field flow fractionation, laser diffraction, microscopic imaging techniques, or disk centrifugation. The average effective particle sizes mentioned herein may be related to weight distributions of the particles. In that instance, by “an average effective particle size of about 150 μm” it is meant that at least 50% of the weight of the particles consists of particles having a particle size of less than the effective average of 50 μm, and the same applies to the other effective particle sizes mentioned. In a similar manner, the average effective particle sizes may be related to volume distributions of the particles but usually this will result in the same or about the same value for the average effective particle size.

The so-called “span” of the particles produced by the process of the invention may be lower than about 3, in particular lower than about 2.5, preferably the span is about 2. Usually the span will not be lower than about 1. As used herein the term “span” is defined by the formula (D₉₀-D₁₀)/D₅₀ wherein D₉₀ is the particle diameter corresponding to the diameter of particles that make up 90% of the total weight of all particles of equal or smaller diameter and wherein D₅₀ and D₁₀ are the diameters for 50 respectively 10% of the total weight of all particles.

Optionally, further excipients may be included in the feed mixture, for example to improve properties of the feed mixture or the resulting solid pharmaceutical composition, such as handling or processing properties. Regardless of whether or not excipients are added to the feed mixture, which obviously results in them being incorporated in the solid dispersion, excipients may also be mixed with the resulting solid pharmaceutical composition during formulation into the desired dosage form.

Excipients suitable for inclusion in the pharmaceutical dosage forms comprise surfactants, solubilizers, disintegrants, pigments, flavors, fillers, lubricants, preservatives, thickening agents, buffering agents, and pH modifiers. In particular, surfactants may be added to further improve solubility of the active agent and may also function as wetting agents. Typical surfactants include sodium lauryl sulphate, polyethoxylated castor oil, e.g. Cremophor EL™, Cremophor RH 40™, Vitamin E TPGS, and polysorbates, such as Tween 20™ and Tween 80™, polyglycolized glycerides such as Gelucire™ 44/14 and Gelucire™ 50/13 (available from Gattefossé, France).

Typical pH modifiers that can be added include acids, such as citric acid, succinic acid, tartaric acid; bases; or buffers.

Prior to use, water or other aqueous media such as those containing ingredients to make the solutions more palatable e.g. sugars, such as glucose or flavors, is added to the powders for reconstitution of the invention. The quantity of water that is added is in the range of about 0.5 ml water per mg TMC125 to about 5 ml, or of about 0.5 ml water per mg TMC125 to about 2 ml, or of about 0.5 ml water per mg TMC125 to about 1 ml, e.g. about 0.6 ml/mg TMC125. Addition of water to a powder of TMC125, dispersed in a water-soluble polymer as specified above, generates a suspension of amorphous TMC125 from which the TMC125 active ingredient does not precipitate, which is unexpected.

The powders for reconstitution of the invention may be used in paediatric applications, not only because of the ease of administration to infants and children but also because of the convenience of dosing in function of age and body weight. Other applications of a formulation that allows flexible dosing, also in adults, is in instances where higher or lower doses or lower doses than provided by a fixed tablet dose is warranted, such as in cases of impaired drug elimination (renal or hepatic failure). A further target group are adult patients that have difficulty in swallowing solid dosage forms such as tablets or capsules. Another advantage is that other anti-HIV agents can be combined with TMC125, by mixing or co-processing with- or in the powders. The powders for reconstitution of the invention show good uptake of the active ingredient and result in good plasma levels, comparable to those obtained with a TMC125 tablet formulation.

The administration of TMC125 as in the present invention may suffice to treat HIV infection, although it may be recommendable to co-administer other HIV inhibitors. The latter preferably include HIV inhibitors of other classes, in particular those selected from NRTIs, PIs, fusion inhibitors, entry inhibitors, integrase inhibitors and maturation inhibitors. In one embodiment, the other HIV inhibitor that is co-administered is a PI. HIV inhibitors that may be co-administered by preference are those used in HAART combinations comprising an NNRTI. For example two further NRTIs or an NRTI and a PI may be co-administered.

In certain instances, the treatment of HIV infection may be limited to only the administration of a powder for reconstitution of TMC125 in accordance with the methodology of this invention, i.e. as monotherapy without co-administration of further HIV inhibitors. This option may be recommended, for example, in cases where the viral load is relatively low, for example where the viral load (represented as the number of copies of viral RNA in a specified volume of serum or plasma) is below about 200 copies/ml, in particular below about 100 copies/ml, more in particular below 50 copies ml, specifically below the detection limit of the viral RNA. In one embodiment, this type of monotherapy is applied after initial treatment with a combination of HIV drugs, in particular with any of the HAART combinations during a certain period of time until the viral load in blood plasma reaches the afore mentioned low viral level.

As used herein the term “treatment of HIV infection” relates to a situation of the treatment of a subject being infected with HIV. The term “subject” in particular relates to a human being. Another application could be the use of TMC125 formulations of the invention to prevent HIV infection in uninfected subjects.

The dose of TMC125 administered, which is determined by the amount of TMC125 in the formulation for use in the invention and the quantity of formulation administered, is selected such that the blood plasma concentration of TMC125 is kept within the blood plasma concentration cut-off values for efficacy and toxicity. The lower value determines efficacy and requires TMC125 to be above a minimum blood plasma concentration. The term “minimum blood plasma level” in this context refers to the lowest efficacious blood plasma concentration, the latter being that blood plasma level that effectively inhibits HIV so that viral load is below the above-mentioned values. The plasma levels of TMC125 should be kept above said minimum blood plasma level to avoid a situation where the viral replication is no longer suppressed, thereby increasing the risk of viruses harboring mutations, which in turn may lead to resistance to the HIV inhibiting drug. Blood plasma levels higher than what is strictly required as minimum level may be preferred to build in a virus-suppressing margin. In one embodiment the minimum blood plasma level is about 25 ng/ml, or 100 ng/ml, or 250 ng/ml. The latter two higher values may be preferred in the instance where a safety margin is desired.

The higher cut-off level, the maximum TMC125 blood plasma level, is determined by side effects and/or toxicity, i.e. plasma concentrations in this high range may induce side effects in patients treated with TMC125. The maximum plasma levels for TMC125 may be as high as 2500 ng/ml or even higher, such as 5000 ng/ml. Optimal plasma levels for TMC125 are therefore within the range as set by the upper and lower cut-off values, in the range from 25 to 5000 ng/ml, or in the range from 100 to 2500 ng/ml. These plasma levels are achieved in HIV-infected subjects taking tablets of TMC125, spray-dried in HPMC, at a dose of 200 mg twice-daily, while mean body weights in these patients were ranging from 65 to 80 kg. For subjects with a considerably higher or lower body weight, such as children, an adapted dose of TMC125 can be given with the present powder for reconstitution. Such a dose may be calculated on the basis of the actual body weight and taking into account the bioavailability of TMC125 as the powder for reconstitution, relative to the tablets mentioned above.

The powders for reconstitution of TMC125 in accordance with the present invention provide effective treatment of HIV infection in that the viral load is reduced while keeping viral replication suppressed. The ease of administration may add to the patients' compliance with the therapy.

When mixed with water, a suspension is formed from which TMC125 does not crystallize. This can be checked using conventional techniques such as X-ray diffraction, Fourier Transform Infrared (FT-IR) and FT-Raman What is formed is a stable suspension of TMC125 containing particles in which the active ingredient stays in the amorphous state. This in turn results in drinkable suspensions that are bioavailable.

Regardless of the TMC125/polymer starting ratio, a constant amount of polymer was found in the residual particles when mixing the powders for reconstitution of the invention with aqueous media. This in particular was the case when using HPMC as water-soluble polymer. Part of the polymer is leached out into the aqueous phase and is believed to have a stabilizing effect on the suspension.

As used herein, the term “about” has its conventional meaning. When used in relation to a numerical value, it may additionally interpreted to cover values that vary within ±20%, or within ±10%, or within ±5%, or within ±2%, or within ±1% of the numerical value.

### EXAMPLES

### Example 1

### Study in Dogs

All formulations contain TMC125 as base.

Composition of the mixtures:

Treatment A: 200 mg TMC125 tablet

| | | |
|---|---|---|
| TMC125 | 200 | mg |
| Hydroxypropylmethylcellulose 2910 5 mPa · s | 600 | mg |
| Polysorbate 20 | 20 | mg |
| Lactose Monohydrate 80 mesh | 180 | mg |
| Microcrystalline Cellulose (PH302) | 212.9 | mg |
| Kollidon CL | 72.8 | mg |
| Colloidal anhydrous silica | 3.9 | mg |
| Hydrogenated cottonseed oil | 10.4 | mg |

Treatment B: 222 mg/g TMC125 spray dried powder

| | |
|---|---|
| TMC125 | 200 mg |
| Hydroxypropylmethylcellulose 2910 5 mPa · s | 600 mg |
| Microcrystalline Cellulose (PH105) | 100 mg |

Treatment C: 200 mg powder formulation containing
spray-dried (taste optimized) TMC125

| | | |
|---|---|---|
| TMC125 | 200 | mg |
| Hydroxypropylmethylcellulose 2910 5 mPa · s | 600 | mg |
| Microcrystalline Cellulose (PH105) | 100 | mg |
| Sorbitol Instant Pharma | 900 | mg |
| Sucralose | 10 | mg |
| Dioctyl sulfosuccinate, sodium salt, 96% | 1 | mg |
| Purified water | 18.19 | mg |

TMC125, HPMC and MCC are spray-dried from an 10% (w/w) ethanol/90% (w/w) dichloromethane mixture. The obtained powder is then mixed with sorbitol, sucralose and DOSS. The aqueous dispersion is made just before administration.

### Bioavailability of Powders for Reconstitution in Dogs

A study was performed to estimate the relative bioavailability (Frel) and to compare the plasma pharmacokinetics of three different powder formulations of TMC125 with a reference tablet (TF035) in dogs at a dose of 200 mg per dog. The reference formulation is the tablet as used in clinical development studies. Nine male beagle dogs, weighing between 9 and 13 kg at the start of the experiment, were included. All nine dogs were dosed the reference tablet (TF035; treatment A). After a wash-out period of 1 week, the dogs were randomly divided in three groups of three animals and were dosed by oral gavage with one of three powders for reconstitution.

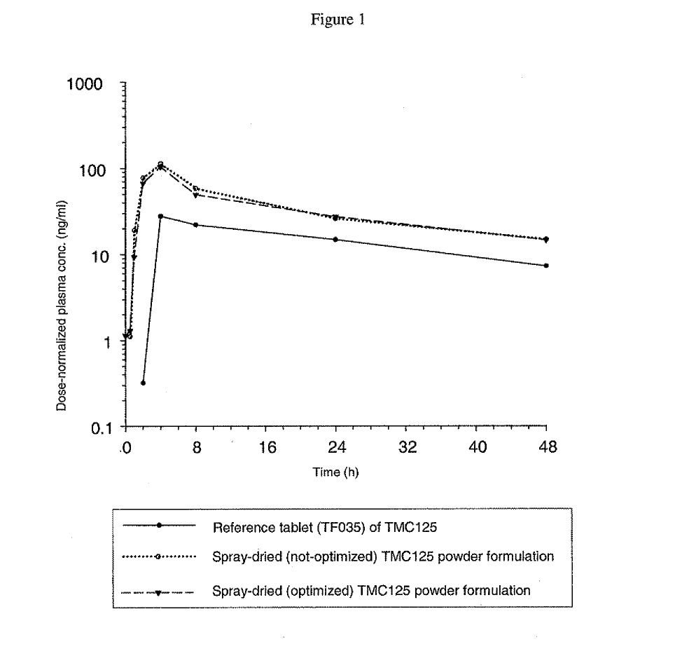

The powders for reconstitution included a powder formulation containing spray-dried TMC125 (HPMC and MCC; treatment B), a powder formulation containing further taste optimized spray-dried TMC125 (sucralose, sorbitol instant and DOSS added; treatment C).The dogs were fasted overnight and, on the days of dosing, were fed a high-fat porridge by oral gavage at approximately 30 min before dosing. No additional food was given when such porridge was provided. Blood samples were taken until 48 h after dosing and plasma concentrations of TMC125 were determined by LC-MS/MS. Results of this study are summarized in the table below and in FIG. 1.

TABLE 1
Mean (n = 3, ± S.D.) pharmacokinetic parameters of TMC125
in male beagle dogs after treatment A (single oral administration
of tablet TF035 containing 200 mg TMC125), serving as
reference for treatment B and C.

| | A | | A | |
|---|---|---|---|---|
| | reference for B² | | reference for C | |
| Treatment | Mean | SD | Mean | SD |
| Cₘₐₓ (ng/ml) | 514 | — | 774 | 293 |
| Tₘₐₓ (h)¹ | 8 | — | 4 | 0 |
| t_{1/2, 24-48 h} (h) | 38 | — | 31 | 17 |
| AUC_{0-48 h} (ng.h/ml) | 8820 | — | 15400 | 4640 |
| AUC_{0-inf} (ng.h/ml) | 13900 | — | 23200 | 6620 |
| AUCₑₓₜᵣₐₚ (%) | 37 | — | 32 | 17 |

¹Median value.
²N = 1; 2 dogs vomited shortly after dosing. No mean or SD calculated.

TABLE 2
Mean (n = 3, ± S.D.) pharmacokinetic parameters of TMC125 in male
beagle dogs after single oral administration of powder formulations
containing spray-dried TMC125 (B), containing spray-dried
(optimized, C) TMC125, at a dose of 200 mg TMC125/dog.

| | B | | C | |
|---|---|---|---|---|
| Treatment | Mean | SD | Mean | SD |
| Cₘₐₓ (ng/ml) | 2190 | 996 | 1920 | 263 |
| Tₘₐₓ (h)¹ | 4 | 0 | 4 | 0 |
| t_{1/2, 24-48 h} (h) | 31 | 16 | 27 | 3 |
| AUC_{0-48 h} (ng.h/ml) | 33500 | 6200 | 29300 | 2670 |
| AUC_{0-inf} (ng.h/ml) | 47600 | 17500 | 39800 | 5450 |
| AUCₑₓₜᵣₐₚ (%) | 27 | 13 | 26 | 3 |
| Frel (%) | 343 | 74 | 185 | 35 |

¹Median value.

FIG. 1: Mean dose-normalised plasma concentrations (ng/ml) versus time profiles of TMC125 in male beagle dogs after single oral administration of a reference tablet TF035 (A), a powder formulation containing spray-dried TMC125 (B), a powder formulation containing spray-dried (optimized, C) TMC125 at a dose of 200 mg TMC125/dog.

The results of the study show favorable pharmacokinetics for the three powders for reconstitution. Considerable inter-individual variability in plasma concentrations was observed, especially after treatment of the reference tablet (TF035). The absorption of TMC125 was relatively slow with Tₘₐₓ observed at 4 hours post-dose after treatment B and C and varying between 2 and 8 hours post-dose after treatment A. Interestingly, administration of TMC125 formulated as a powders for reconstitution resulted in higher exposure compared to the reference tablet (TF035). The dose-normalized (for body weight adjustment) Cₘₐₓ values after treatment B and C were on average 3.5, 2.5 and 2.6 times higher than after treatment A. The average relative bioavailability of the three powder formulations relative to the reference tablet (TF035) was 343, 185 and 236% for treatment B and C, respectively. No major differences were observed in the pharmacokinetics using not-optimized vs optimized TMC125 in the spray-dried powder formulations.

### Example 2

### Human Study

A Phase I, open-label, randomized, single-dose, 3-period crossover trial in 24 healthy subjects was performed to evaluate the relative oral bioavailability of a single dose of 200 mg TMC125 spray-dried product formulated as 2 batches of a powder formulation (F051) compared to the 100-mg tablet (F060) after oral administration in the fed state.

The F060 100-mg tablet of TMC125 is a solid dispersion containing the active pharmaceutical ingredient in a stabilized amorphous form.

The F051 powder formulation of TMC125 was also manufactured by spray-drying technology. The 2 batches of the F051 powder formulation (Powder 1 and Powder 2) were both included in this comparison to ensure the manufacturing process did not influence the pharmacokinetics of the powder formulation after oral administration with food in healthy subjects.

The 800-mg b.i.d. (formulation TF035, used in the dog study above) dose of TMC125 has been determined to be a relevant, effective, and well-tolerated dose. Since 200 mg b.i.d. of formulation F060 produced an exposure comparable to that of 800 mg b.i.d. of formulation TF035, 200 mg b.i.d. of formulation F060 has been designated as a dose to be further explored in the future clinical development of TMC125.

Subjects were randomly assigned to 1 of 6 possible treatment sequences and received all treatments (1 in each period). There was a washout period of at least 14 days between intakes. A full pharmacokinetic profile of TMC125 was determined over 96 hours for each formulation.

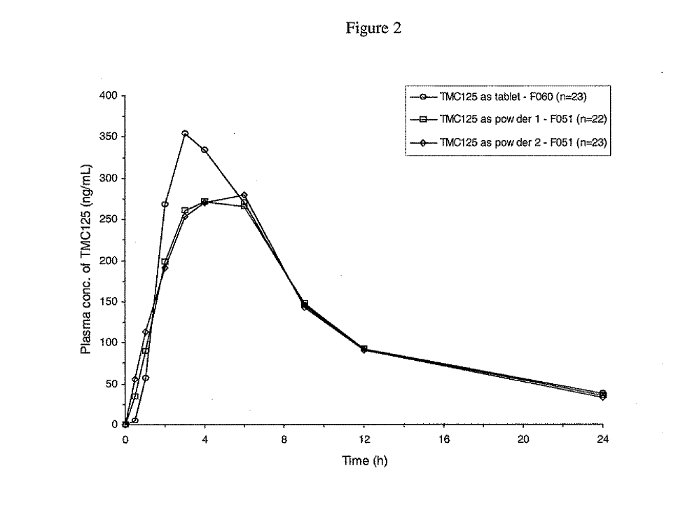

The results are summarized in the Tables below and in FIG. 2.

TABLE 3
Mean Pharmacokinetic Parameters for TMC125 after a Single Dose of
200 mg TMC125 as a Tablet and Two Powders for Reconstitution

| | TMC125 as 100-mg | TMC125 as | TMC125 as |
|---|---|---|---|
| | Tablet-F060 | Powder 1-F051 | Powder 2-F051 |
| | (reference) | (test 1) | (test 2) |
| n | 23 | 22 | 23 |
| Cₘₐₓ, ng/mL | 422.2 ± 166.3 | 312.5 ± 134.3 | 328.3 ± 138.8 |
| t_{tag}, h | 0.0 (0.0-1.0) | 0.0 (0.0-0.0) | 0.0 (0.0-0.0) |
| tₘₐₓ, h | 3.0 (2.0-6.0) | 5.0 (2.0-6.0) | 4.0 (1.0-6.0) |
| AUCₗₐₛₜ, ng.h/mL | 4766 ± 2916 | 4361 ± 2768 | 4254 ± 2509 |
| AUC_{□}, ng.h/mL^{a} | 6105 ± 4646 | 5528 ± 4470 | 5223 ± 3561 |
| t_{1/2term}, h^{a} | 58.35 ± 21.44 | 53.92 ± 20.25 | 54.42 ± 23.56 |

Values are (mean ± SD, tₘₐₓ/t_{tag}: median [range])
^{a}Accurate determination not possible

TABLE 4
Summary of the Statistical Analysis of the Pharmacokinetic Parameters of
TMC125: Powder 1 Compared to 100-mg tablet.

| | LSmeans^{a} | | | | | |
|---|---|---|---|---|---|---|
| | TMC125 as | TMC125 as | | | | |
| | 100-mg | Powder 1 - | | | | |
| | Tablet - F060 | F051 | LSmeans | | p-value | |
| Parameter | (reference) | (test 1) | ratio, % | 90% CI, %^{b} | Period | Sequence |
| Cₘₐₓ, ng/mL | 391.4 | 293.7 | 75.04 | 68.25-82.49 | 0.0528 | 0.3658 |
| AUCₗₐₛₜ, | 4178 | 3751 | 89.78 | 84.71-95.16 | 0.1287 | 0.1780 |
| ng · h/mL | | | | | | |
| | Median | | | | | |
| | TMC125 as | TMC125 as | | | | |
| | 100-mg | Powder 1 - | Treatment | | | |
| | Tablet - F060 | F051 | difference | | p-value | |
| Parameter | (reference) | (test 1) | median | 90% CI, %^{b} | Period | Sequence |
| t_{lag}, h | 0.25 | 0.0 | −0.25 | (−0.5)-(−0.25) | 0.0564 | 0.0564* |
| tₘₐₓ, h | 3.0 | 5.0 | 0.5 | (0.0)-(1.5) | 0.5374 | 0.6646 |

^{a}n = 23 for Treatment A (reference) and n = 22 for Treatment B (test)
^{b}90% confidence intervals.
*Statistically significant difference

FIG. 2: Mean (n=24) Plasma Concentration-Time Curves of a Single Dose of 200 mg TMC1.25 for 3 Different Formulations in Human Healthy Volunteers

The 90% confidence intervals for AUCₗₐₛₜ were within the predefined no-effects limits of 80-125% when comparing Powder 1 or 2 with the 100-mg tablet. The LS means (90% confidence intervals) for Cmax and AUClast of TMC125 were 75.04% (68.25-82.49) and 89.78% (84.71-95.16), respectively, for Powder 1; and 76.70% (67.78-86.80) and 89.46% (84.03-95.24), respectively, for Powder 2, compared to those of the 100-mg tablet. There were no treatment effects for Cₘₐₓ and AUCₗₐₛₜ and AUCₗₐₛₜ of TMC125 when Powder 2 was compared to Powder 1. The 90% confidence intervals of the LS means ratios were within the predefined no-effect limits of 80-125% for both Cₘₐₓ and AUCₗₐₛₜ.

Administration of single doses of 200 mg TMC125 in healthy subjects was generally safe and well tolerated. No clinically relevant safety issues were identified with respect to the parameters studied, including AEs, laboratory and cardiovascular safety, and physical and skin examinations.

The results of the present trial demonstrate that the extent of exposure to TMC125 after administration of a single dose of 200 mg TMC125 formulated as Powder 1 or Powder 2 was slightly lower than after administration of TMC125 as the 100-mg tablet in the presence of food. Interestingly, the lower bioavailability is primarily determined by a lower Cₘₐₓ, whereas Cₘᵢₙ is predicted to be similar for all 3 treatments. As the latter is related to efficacy, overall therapeutic efficacy with the powders for reconstitution should be the same.

1. A drinkable formulation comprising an aqueous phase with a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, and a hydroxyalkyl alkylcellulose.

2. A drinkable formulation comprising particles, wherein the particles comprise TMC125, or a pharmaceutically acceptable acid-addition salt thereof, dispersed in a water-soluble polymer selected from polyvinylpyrrolidone, a copolymer of vinylpyrrolidone and vinyl acetate, and a hydroxyalkyl alkylcellulose, suspended in an aqueous phase.

3. The formulation according to claim 1, wherein the aqueous phase is water, optionally containing a further ingredient such as a flavor, a colorant, a sweetener, a taste making agent.

4. The formulation according to claim 2, wherein the aqueous phase contains part of the water-soluble polymer in solution.

5. The formulation according to claim 1, wherein in the particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1.

6. The formulation according to claim 1, wherein in the particles comprising TMC125, or a pharmaceutically acceptable acid-addition salt thereof, dispersed in the water-soluble polymer, the weight by weight ratio between TMC125 and the polymer is about 2:1.

7. A particle comprising TMC125, or a pharmaceutically acceptable acid-addition salt thereof, dispersed in the water-soluble polymer, the weight by weight ratio between TMC125 and the polymer is in the range of about 2.5:1 to about 1.5:1.

8. The particle according to claim 7, comprising TMC125, or a pharmaceutically acceptable acid-addition salt thereof, dispersed in the water-soluble polymer, the weight by weight ratio between TMC125 and the polymer is in the range of about 2:1.

9. A process for preparing a drinkable formulation according to any of claims 2-6, wherein water is mixed with a powder comprising TMC125, or a pharmaceutically acceptable acid-addition salt thereof, dispersed in a water-soluble polymer selected from polyvinylpyrrolidone, a copolymer of vinylpyrrolidone and vinyl acetate, and a hydroxyalkyl alkylcellulose.

10. The A process for preparing a drinkable formulation of TMC125, said process comprising mixing a powder comprising-TMC125, or a pharmaceutically acceptable acid-addition salt thereof, dispersed in a water-soluble polymer selected from polyvinylpyrrolidone, a copolymer of vinylpyrrolidone and vinyl acetate, and a hydroxyalkyl alkylcellulose, with water.

11. The process according to claim 10, wherein the water-soluble polymer is selected from polyvinylpyrrolidone, a copolymer of vinylpyrrolidone and vinyl acetate, and a hydroxyalkyl alkylcellulose.

12. The process according to claim 10, wherein the hydroxyalkyl alkylcellulose is hydroxypropyl methylcellulose.

13. The process according to claim 10, wherein the hydroxyalkyl alkylcellulose is HPMC 2910 5 mPa·s.

14. The process according to claims 10-13, wherein the powder comprising TMC125 is obtained by spray drying.

15. A suspension of amorphous TMC125, or a pharmaceutically acceptable acid-addition salt thereof, and a water-soluble polymer selected from polyvinylpyrrolidone, a copolymer of vinylpyrrolidone and vinyl acetate, and a hydroxyalkyl alkylcellulose, in an aqueous medium.

16. A suspension of amorphous TMC125 according to claim 15, obtainable by adding water to a powder comprising an anti-virally effective amount of TMC125, or a pharmaceutically acceptable acid-addition salt thereof, dispersed in a water-soluble polymer selected from polyvinylpyrrolidone, a copolymer of vinylpyrrolidone and vinyl acetate, and a hydroxyalkyl alkylcellulose.

17. A suspension of amorphous TMC125 according to claim 15, wherein the water-soluble polymer is selected from polyvinylpyrrolidone, a copolymer of vinylpyrrolidone and vinyl acetate, and a hydroxyalkyl alkylcellulose.

18. A suspension of amorphous TMC125 according to claim 15, wherein the hydroxyalkyl alkylcellulose is hydroxypropyl methylcellulose.

19. A process for preparing a suspension of amorphous TMC125, said process comprising adding water to TMC125, dispersed in a water-soluble polymer selected from polyvinylpyrrolidone, a copolymer of vinylpyrrolidone and vinyl acetate, and a hydroxyalkyl alkylcellulose.

20. The formulation according to claim 2, wherein the aqueous phase is water, optionally containing a further ingredient such as a flavor, a colorant, a sweetener, a taste making agent.

21. The formulation according to claim 3, wherein the aqueous phase contains part of the water-soluble polymer in solution.

22. The formulation according to claim 2, wherein in the particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1.

23. The formulation according to claim 3, wherein in the particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1.

24. The formulation according to claim 4, wherein in the particles comprising TMC125, or a pharmaceutically acceptable acid-addition salt thereof, dispersed in the water-soluble polymer, the weight by weight ratio between TMC125 and the polymer is in the range of about 2.5:1 to about 1.5:1.

25. The formulation according to claim 2, wherein in the particles comprising TMC125, or a pharmaceutically acceptable acid-addition salt thereof, dispersed in the water-soluble polymer, the weight by weight ratio between TMC125 and the polymer is about 2:1.

26. The formulation according to claim 3, wherein in the particles comprising TMC125, or a pharmaceutically acceptable acid-addition salt thereof, dispersed in the water-soluble polymer, the weight by weight ratio between TMC125 and the polymer is about 2:1.

27. The formulation according to claim 4, wherein in the particles comprising TMC125, or a pharmaceutically acceptable acid-addition salt thereof, dispersed in the water-soluble polymer, the weight by weight ratio between TMC125 and the polymer is about 2:1.

28. A suspension of amorphous TMC125 according to claim 18, wherein the hydroxypropyl methylcellulose is HPMC 2910 5 mPa·s.
